(19) United States
(12) Patent Application Publication
(10) Pub. No.: US 2022/0106087 A1
(43) Pub. Date: Apr. 7, 2022

(54) FLUID DISTRIBUTOR AND REPLACEMENT CARTRIDGE

(71) Applicant: CAMBLI TECHNOLOGIES INC., St-Jean-sur-Richelieu (CA)

(72) Inventors: Martin COUSINEAU, St-Jean-sur-Richelieu (CA); Simon CHICOINE, Marieville (CA); Jean-Philippe COMEAU, Ville Sainte-Catherine (CA)

(21) Appl. No.: 17/240,674

(22) Filed: Apr. 26, 2021

Related U.S. Application Data
(60) Provisional application No. 63/086,106, filed on Oct. 1, 2020.

(51) Int. Cl. B65D 47/20; B65D 3/26; B65D 35/56; B65D 79/02; B65D 83/00; B65D 83/20; A61L 2/26; A61L 2/00

(52) U.S. Cl. CPC B65D 47/20; B65D 3/268; B65D 35/56; B65D 79/02; A61L 2202/15; B65D 83/20; A61L 2/26; A61L 2/0088; B65D 83/0055

(57) ABSTRACT

A cartridge for a fluid distributer, including a cartridge housing, the bottom wall being arranged at an angle to the front wall less than 90 degrees; a fluid-containing package including a bag containing a fluid product in an interior of the bag. A fluid distributor including: a distributor housing including a door covering a portion of a front side of the housing, the front wall or the door including a window; a receiving surface extending rearward and upward from the front wall, the housing forming a cartridge receiving recess, a front surface of the cartridge being visible through the window when installed therein, the front wall and the receiving surface being configured to match a form of the cartridge; an operating member operatively connected to a pressure member configured to apply pressure to the dispensing tube upon rotation of the operating member when the cartridge is installed in the distributor.

### CROSS-REFERENCE

The present application claims priority to U.S. Provisional Patent Application No. 63/086,106, entitled “Fluid Distributor and Replacement Cartridge,” filed Oct. 1, 2020, the entirety of which is incorporated herein by reference.

### FIELD OF THE TECHNOLOGY

The present technology relates to fluid distributors and their corresponding cartridges, more specifically to hand sanitizer distributors and replacement cartridges thereof.

### BACKGROUND

It has become increasingly common to provide installations for members of the public clean or sanitize their hands before entry into indoor public spaces, such as shops, restaurants, offices, libraries, etc. Many such locations choose to provide hand sanitizing gel or liquid, as it does not require the infrastructure necessary for a hand-washing station (i.e. a sink with running water).

Hand sanitizer distributors are often provided, which hold relatively large quantities of hand sanitizer and dispense smaller amounts thereof to users. In order to reliably provide hand sanitizer for each member of the public entering a given location, distributors must be regularly refilled. Some distributors are refilled by pouring additional hand sanitizing product into a reservoir in the distributor. In such distributor, the apparatus may require regular cleaning, as adding new product directly into the distributor may result in old product mixing with newly added product.

One approach to overcome the above disadvantage is configuring distributors to receive refill or replacement cartridges of hand sanitizing product instead. In some cases, this can result in additional labor for operators of the distributor, as the level of remaining hand-sanitizing product in the cartridge must be checked regularly to ensure that the distributor does not run out of product.

There is thus a need for hand sanitizer distributors and/or replacement cartridges that overcome at least some previously known disadvantages.

### SUMMARY

It is an object of the present technology to ameliorate at least some of the inconveniences present in the prior art.

According to one aspect of the present technology, there is provided a distributor configured to receive a refill or replacement cartridge containing a fluid product to be dispensed by the distributor. In the embodiment illustrated herein, the distributor operates entirely by mechanical assembly, wherein an end user pivots a rigid member connected by cams to an assembly that causes fluid product to be dispensed to the end user. As such, the distributor does not require batteries that would need to be regularly replaced, nor does the distributor need to be located in proximity to a power supply or outlet in order to operate.

For distributors of the prior art which are refilled directly into a reservoir in the distributor, the end user has no way of knowing if the product being dispensed is safe and/or effective. The distributor according to the present technology, however, defines a cartridge receiving recess therein where bottom and top surfaces defining the recess and top and bottom surfaces of the cartridge are similarly angled. The matching forms of the cartridge and recess in the distributor aid in assuring that only cartridges of the prescribed shape are used, preventing an operator from inserting into the distributor different cartridges that may not meet safety standards, etc.

The cartridge according to the present technology further includes identifying information presented on a front surface thereof, relating to the fluid product contained in the cartridge. The distributor further includes a transparent window configured to align with the front side of the cartridge when the cartridge is installed therein. The identifying information is thus visible to the end user of the distributor during use. This allows the end user to know what fluid product is dispensed by the distributor, as well as key components of the fluid product, such a possible allergens, scents, etc.; this also further aids in ensuring that the operator cannot replace the approved product with another product of unknown safety or origin, as the end user sees when the identifying information is missing.

The cartridge according to the present technology further includes a dispensing tube connected thereto for aiding in maintaining safety and cleanliness of the distributor. As the fluid product passes through the dispensing tube to the end user, no portion of the distributor comes into contact with the fluid product. As the tube is connected to the cartridge, replacement of the cartridge includes replacement of the dispensing tube. Thus no old product remains in the distributor when removing an old cartridge and inserting a new cartridge. Replacement of the cartridge can thus be performed without additional cleaning of the distributor, and no contamination of the fluid product is incurred from previous refills of the distributor. Replacement of the dispensing tube with the cartridge can further aid in maintaining longevity of the distributor, as some dispensing tubes (when installed directly in the distributor) can become defective over time.

The cartridge according to the present technology also includes a removable portion of the housing, such that when the cartridge is installed in the distributor, the level of fluid product in the cartridge is visible from a front side of the cartridge. As the transparent window of the distributor is configured to align with the front side of the cartridge, the operator of the distributor can easily inspect the remaining level of fluid product in the distributor without opening or otherwise manipulating the distributor or cartridge. The distributor according to the present technology is also configured to distribute a calibrated dose of fluid product from the cartridge at each utilization of the distributor. As such in some cases, the operator can further gage when the cartridge needs to be replaced based on the number of utilizations and/or the number of end users having used the distributor. In some embodiments, the number of doses contained in the cartridge may be included in the identifying information. In some embodiments, the distributor could include a counter mechanism (either electronic or mechanical) to count the number of doses used and/or remaining.

In some cases, arrangement of the angles of the bottom of the cartridge and the recess further aid in more completely emptying the fluid product from the cartridge. The angled arrangement creates a tilt of the bottom portion of a bag of the fluid product within the cartridge, such that the fluid product is forced toward the dispensing tube by gravity. In cases where the fluid product is a gel, the higher viscosity can in some cases impede the fluid to fully flow from cartridges of the prior art where bottom surfaces are more horizontally arranged.

According to another aspect of the present technology, there is provided a cartridge for a fluid distributer, including a cartridge housing including: a front wall, a right wall extending rearward from a right edge of the front wall, a left wall extending rearward from a left edge of the front wall, a rear wall extending between a rear edge of the right wall and a rear edge of the left wall, and a bottom wall extending rearward and upward from the front wall, the bottom wall being arranged at an angle to the front wall, the angle being less than 90 degrees; a fluid-containing package disposed in the housing, the fluid-containing package including a bag containing a fluid product in an interior of the bag.

In some embodiments, the fluid-containing package further includes a dispensing tube fluidly communicating with the interior of the bag.

In some embodiments, at least one of the front wall and the bottom wall is perforated to allow for selective removal of a removable portion of the at least one of the front wall and the bottom wall; and when the removable portion is removed, the fluid-containing package being configured to allow the dispensing tube to be selectively extended outward and downward from the cartridge housing.

In some embodiments, the removable portion is defined by perforations of both the front wall and the bottom wall.

In some embodiments, the removable portion is further configured to allow a level of remaining fluid in the bag to be visible from an exterior of the cartridge.

In some embodiments, the front wall displays identifying information related to the fluid product of the fluid-containing package; and the identifying information is arranged on the front wall such that the identifying information is visible to a user through a window when the cartridge is installed in the fluid distributor.

In some embodiments, the angle is measured between an inner surface of the front wall and an inner surface of the bottom wall; and the angle is greater than 60 degrees.

In some embodiments, the angle is approximately 75 degrees.

In some embodiments, the cartridge housing further includes a top wall extending rearward and upward from the front wall, the top wall being arranged at a top wall angle to the front wall; and the top wall angle is greater than 90 degrees.

In some embodiments, the fluid product is a hand sanitizing gel.

In some embodiments, the cartridge housing is formed from a folded sheet of rigid paper-based material.

According to yet another aspect of the present technology, there is provided a fluid distributor, including: a distributor housing including: a front wall, at least one other wall connected to the front wall, and a door pivotably connected to one of the front wall and the at least one other wall, the door selectively allowing access to an interior of the distributor housing, the door covering a portion of a front side of the housing, at least one of the front wall and the door including a transparent window; a receiving surface disposed in the distributor housing, the receiving surface extending rearward and upward from an interior of the front wall, the front wall, the door, the at least one other wall, and the receiving surface forming a cartridge receiving recess for selectively receiving a fluid-containing cartridge therein, a front surface of the cartridge being visible through the transparent window when installed therein, the front wall and the receiving surface being configured to match a form of the cartridge; a passage being defined within the distributor housing for receiving a dispensing tube of the cartridge, the passage being in fluid communication with the recess and an exterior of the housing; a pressure member disposed adjacent to the passage, the pressure member being selectively moveably into and out of the passage; and an operating member extending at least partially from the housing, the operating member being operatively connected to the pressure member such that the pressure member moves in the passage upon movement of the operating member by a user, the pressure member being configured to apply pressure to the dispensing tube upon rotation of the operating member when the cartridge is installed in the distributor.

In some embodiments, the receiving surface is arranged at a surface angle to the front wall; and the surface angle is greater than 60 degrees.

In some embodiments, the surface angle is approximately 75 degrees.

In some embodiments, the distributor further includes a base; and wherein: the distributor housing is connected to and extends upward from the base, and the operating member is a foot pedal extending from a lower portion of the distributor housing.

In some embodiments, the distributor housing is configured for mounting to a wall; the operating member is a pivotable member extending from the distributor housing.

In some embodiments, the receiving surface defines a hole therein, the cartridge receiving recess being fluidly connected to the passage through the hole; and when the cartridge is installed in the cartridge receiving recess, the dispensing tube of the cartridge extends through the hole and into the passage.

In some embodiments, the operating member is mechanically connected to the pressure member.

In some embodiments, the pressure member is configured to translate generally horizontally into and out of the passage in response to pivoting of the operating member by the user; and when the cartridge is installed in the cartridge receiving recess, translation of the pressure member causes the dispensing tube of the cartridge to be compressed and eject from the dispensing tube a predetermined amount of fluid product from the cartridge.

In some embodiments, the distributor further includes a locking assembly disposed in the door for selectively securing the door to prevent access to the cartridge receiving recess.

In some embodiments, the at least one other wall includes a rear wall generally parallel to the front wall; a left wall extending from a left side of the front wall to a left side of the rear wall; and a right wall extending from a right side of the front wall to a right side of the rear wall.

In some embodiments, the door is pivotably connected to the front wall; the transparent window is disposed in the door; and when in a closed position, the door extends upward, and then rearward and upward from a top edge of the front wall to a top edge of the rear wall.

In some embodiments, the front wall, the left wall, and the right wall define a hand-receiving recess on an exterior side of the distributor housing; the passage is further fluidly connected to the hand-receiving recess; and when the cartridge is installed in the cartridge receiving recess, the dispensing tube extends through the passage.

In some embodiments, the distributor further includes the cartridge disposed in the cartridge receiving recess, the cartridge including a cartridge housing including: a front wall, a right wall extending rearward from a right edge of the front wall, a left wall extending rearward from a left edge of the front wall, a rear wall extending between a rear edge of the right wall and a rear edge of the left wall, and a bottom wall extending rearward and upward from the front wall, the bottom wall being arranged at an angle to the front wall; a fluid-containing package disposed in the housing, the fluid-containing package including: a bag containing a fluid product in an interior of the bag, and the dispensing tube fluidly communicating with the interior of the bag; and wherein: the receiving surface and the front wall of the distributer housing are arranged to support the cartridge in a generally vertical orientation, the angle between the bottom wall and front wall of the cartridge housing being approximately equal to the angle between the receiving surface and the front wall of the distributor housing.

In some embodiments, the receiving surface and the bottom wall of the cartridge housing are arranged at angles chosen to provide optimal flow for a viscosity value of the fluid product contained in the bag of the fluid-containing package.

For purposes of this application, the term “fluid” is meant to be understood as any material of flowing consistency that can be distributed through a tube, including but not limited to liquids, gels and gel-like materials. The fluids discussed in relation to the present technology include, but are not limited to: hand sanitizing gel, hand sanitizing liquid, cleaning or disinfecting liquid, and liquid soap.

In the present context, the term “operator” is intended to refer to a person generally responsible for maintaining function of the distributor. While the operator could be an owner of the distributor, “operator” is meant to also signify any employee or person otherwise charged with ensuring continued operation of the distributor.

In the present context, the term “end user” is intended to refer to a person seeking to receive and/or use some quantity of the fluid product contained in the cartridge and dispensed by the distributor, but not having authorization, interest, etc. in removing or otherwise handling the cartridge.

For purposes of this application, terms related to spatial orientation such as front, rear, top, bottom, left, and right, are as they would normally be understood with respect to the point of view of the distributor. Terms related to spatial orientation when describing or referring to components or sub-assemblies of the distributor, separately from the distributor, such as a replaceable cartridge for example, should be understood as they would be understood when these components or sub-assemblies are mounted in the distributor, unless specified otherwise in this application.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein. The explanations provided above regarding the above terms take precedence over explanations of these terms that may be found in any one of the documents incorporated herein by reference.

Additional and/or alternative features, aspects and advantages of embodiments of the present technology will become apparent from the following description, the accompanying to drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a top, front, right side perspective view of a fluid distributor according to the present technology;

FIG. 2 is a partially-transparent, close-up, top, front, right side perspective view of the fluid distributor of FIG. 1;

FIG. 3 is a close-up, top, front, left side perspective view of the fluid distributor of FIG. 1 with a cartridge installed therein, with a door is an open position;

FIG. 4 is the perspective view of the fluid distributor and cartridge of FIG. 3, with the cartridge partially removed;

FIG. 5 is a front elevation view of the cartridge of FIG. 3;

FIG. 6 is a bottom, front, right side perspective view of the cartridge of FIG. 5;

FIG. 7 is a left side elevation view of the cartridge of FIG. 5;

FIG. 8 is the front elevation view of FIG. 5, with a portion of the housing removed and a fluid-containing package of the cartridge being deployed;

FIG. 9 is a front elevation view of another embodiment of a distributor in accordance with the present technology;

FIG. 10 is a rear elevation view of the distributor of FIG. 9;

FIG. 11 is a top, front, left side perspective view of the distributor of FIG. 9; and

FIG. 12 is a top plan view of the distributor of FIG. 9.

It should be noted that the Figures may not be drawn to scale, except where otherwise noted.

### DETAILED DESCRIPTION

The present technology is described herein with respect to a fluid distributor 100. In the present examples, the fluid distributor 100 is a distributor 100 for hand sanitizing gel. It is contemplated that the distributor 100 could be configured for distributing different fluids, including but not limited to: hand sanitizing liquids, hand cleaning products, lotion, hydrating cream, other products designed to be applied to or used hands, and liquid soaps, such as dishwashing soap, shampoo, or shower gel.

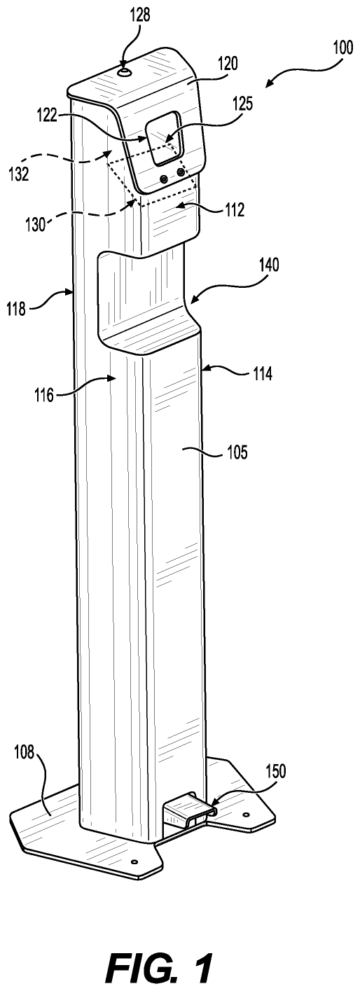

An embodiment of the fluid distributor 100, also referred to herein as the distributor 100, is illustrated in FIGS. 1 to 4.

The distributor 100 includes a distributor housing 105 which forms the overall structure of the distributor 100. The distributor 100 also includes a base 108 to allow the distributor 100 to rest in a stable position on the ground. The distributor housing 105 is connected to and extends upwards from the base 108. In some embodiments, the housing 105 could be integrally formed with the base 108. It is also contemplated that the housing 105 could include additional features for rest directly on the ground, for example with rubber feet connected to bottom edges of the housing 105. The housing 105 and the base 108 are formed from aluminum, but different materials could be used, including, but not limited to, different metals and/or alloys including stainless steel and plastic, as well as combinations thereof. In some embodiments the distributor 100 could be formed by extrusion or integrally formed.

The distributor housing 105 includes a front wall 112, a left wall 114, a right wall 116, and a rear wall 118. The front, left, right, and rear walls 112, 114, 116, 118 are integrally connected. It is contemplated that the one or more of the walls 112, 114, 116, 118 could be fastened, glued or otherwise connected together depending on the embodiment. It is also contemplated that the distributor housing 105 could include more or fewer walls, depending on design or aesthetic choices. For example, the distributor housing 105 could be formed from a front wall and a semi-circular second wall attached thereto.

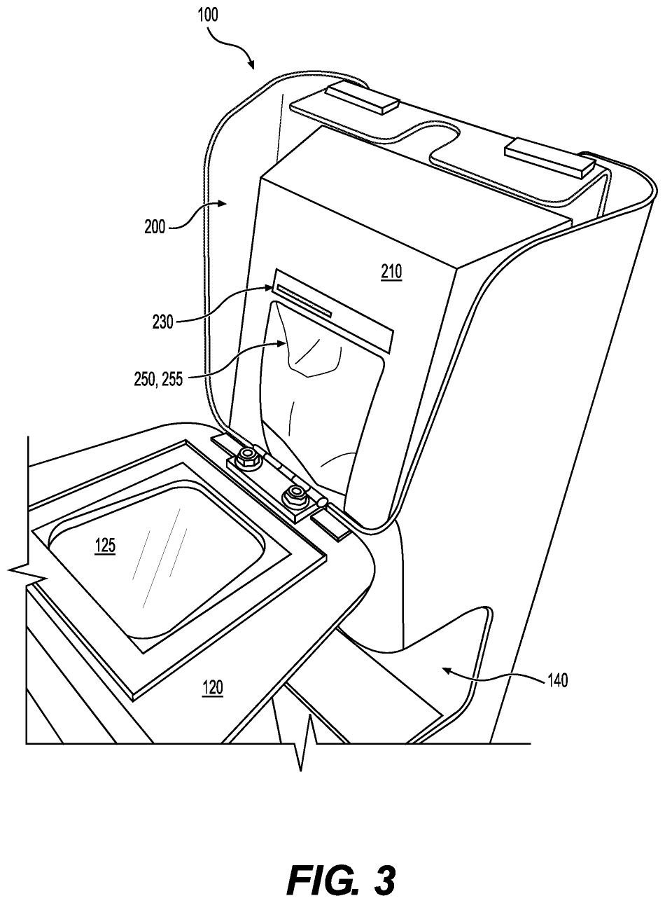

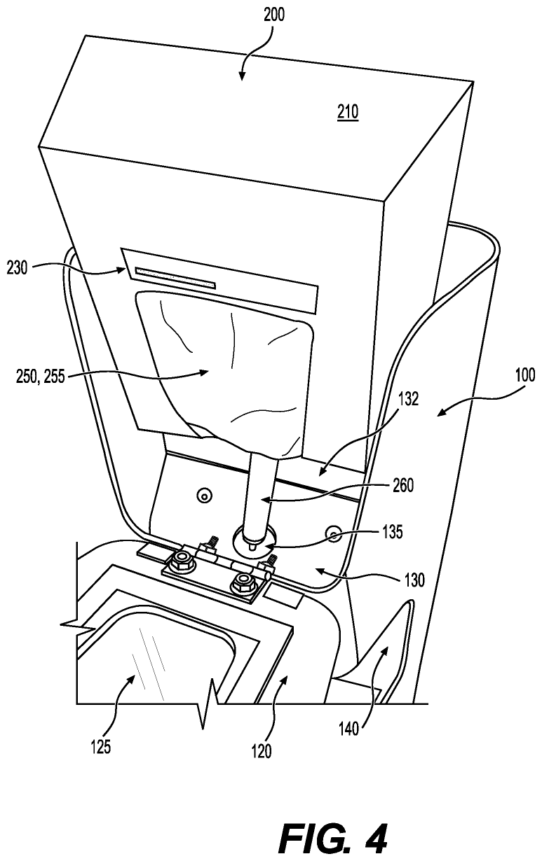

The distributor housing 105 further includes a door 120 pivotably connected to of the front wall 112. The door 120 extends upward from the front wall 112 and extends over a top of the housing 105. The door 120 covers a portion of a front side of the distributor housing 105. Depending on the embodiment, the door 120 could cover a larger or smaller portion of the front side of the distributor housing 105. The door is illustrated in a closed position in FIG. 1. As is illustrated in FIGS. 3 and 4, the door 120 selectively pivots forward and downward to open away from the rear wall 118 to an open position. The door 120 provides selective access to an interior of the distributor housing 105, as will be described in more detail below. In some embodiments, the door 120 could be pivotably connected to a top edge of the rear wall 118, where the door 120 could cover extend over a top of the housing 105 and pivot upward to allow access to an interior of the distributor housing 105.

The door 120 defines an aperture 122 therein. A transparent window 125 is disposed in the aperture 122 to allow a user to see into the interior of the distributor housing 105. In some embodiments, the transparent window 125 could be excluded and the user could see into the interior through the open aperture 122. The transparent window 125 is formed from a flat plastic plate, but it is contemplated that glass could also be used. Depending on the size and arrangement of the door 120, it is also contemplated that the aperture 122 could be defined by the front wall 112 in some embodiments (the transparent window 125 being disposed in the aperture 122 in the front wall 112 in embodiments including the transparent window 125).

The door 120 also includes a locking mechanism 128 on its rear edge. The locking mechanism 128 cooperates with the rear wall 118 to selectively secure the door 120 in its closed position. The locking mechanism 128 prevents unauthorized access to the interior of the distributor housing 105, as will be described in more detail below.

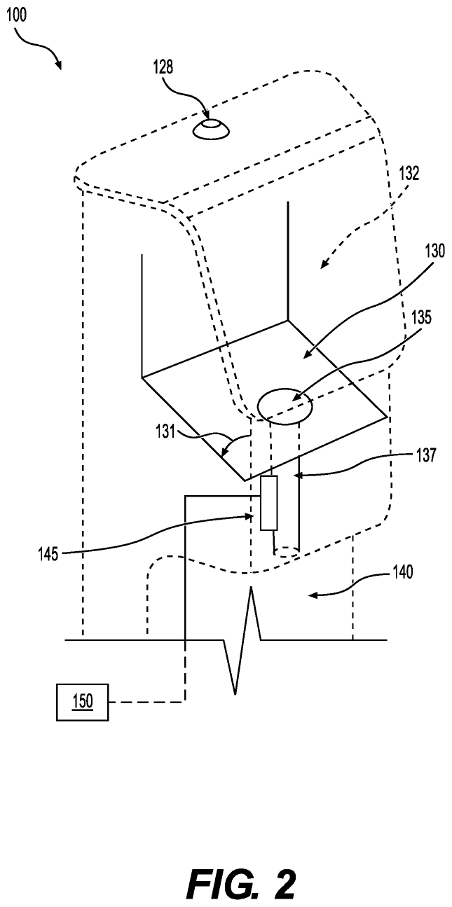

The distributor 100 further includes a receiving surface 130 disposed within the housing 105, illustrated in the partially-transparent view of the distributor 100 in FIG. 2. The receiving surface 130 extends rearward and upward from an interior of the front wall 112. Specifically, the receiving surface 130 is arranged at a surface angle 131 relative to the front wall 112 (measured from the interior of the front wall 112 to a to surface of the receiving surface 130) which is less than 90 degrees. As is schematically illustrated in FIG. 2, the angle 131 is approximately 75 degrees and more generally greater than 60 degrees from vertical. The receiving surface 130 is formed from metal, but different materials could be used including, but not limited to, plastic. In some embodiments, the receiving surface 130 could be selectively removable from the housing 105 to allow maintenance or upgrades to the distributor 100.

The receiving surface 130 and the front, left, right, and rear walls 112, 114, 116, 118 of the housing 105 define a receiving recess 132 in the interior of the distributor 100. As will be described in more detail below, the recess 132 is shaped and arranged to receive therein a fluid-containing cartridge 200, which provides the hand-sanitizing gel (or other fluid) to be distributed by the fluid distributor 100.

The receiving surface 130 defines a hole 135 for extending a portion of the cartridge 200 therethrough. Specifically, the hole 135 fluidly connects with a passage 137 that extends through an interior of the housing 105 and fluidly connects with an exterior of the housing 105. Below the passage 137, the front, left, and right walls 112, 114, 116 further define a hand receiving recess 140 on a front side of the distributor 100. A bottom of the passage 137 is open in a top side of the hand receiving recess 140, such that a user can place their hands below the passage 137 to receive the fluid product therefrom, as will be described in more detail below.

The distributor 100 further includes a pressure member 145 disposed adjacent to the passage 137. The pressure member 145 in the present embodiment has a generally vertical face for applying pressure to the dispensing tube of the cartridge 200, by squeezing the tube by a volume corresponding to a desired volume of fluid to be dispensed. The pressure member 145 is selectively moveable to squeeze the dispensing tube in the passage 137. The distributor 100 further includes an operating member 150 operatively connected to the pressure member 145. In the present embodiment, the operating member 150 is a foot pedal 150 extending partially from a lower portion of the housing 105. In some embodiments, the operating member 150 could be implemented as a handle protruding from an upper portion of the housing 105 (for use with a user's arm, for example). Upon pivoting of the foot pedal 150 by the user, the pressure member 145 displaces into the passage 137 to press on the dispersing tube of the cartridge 200, as will be described in more detail below. In some embodiments, the pressure member 145 could translate or rotate depending on the particular case. It is also contemplated that the distributor 100 could include one or more mechanisms for adjusting the amount of fluid product dispensed.

In the present embodiment, the foot pedal 150 is connected to the pressure member 145 by a series of mechanical cams (not shown), such that the pressure member 145 moves in and out the passage 137 upon movement of the foot pedal 150 by a user. By connecting the foot pedal 150 to the pressure member 145 mechanically, the distributor 100 does not need an electrical power source. As such, the distributor 100 does not need batteries (which would need to be replaced regularly by the operator) nor is the distributor 100 required to be located in the vicinity of an electrical outlet. In some embodiments, of course, electrical connections and/or actuators, including between the operating member 150 and the pressure member 145, could be included depending on design details. It is further contemplated that the distributor 100 could include fluid level indicators, Bluetooth™ or other wireless connection technology, or various electronic accessories.

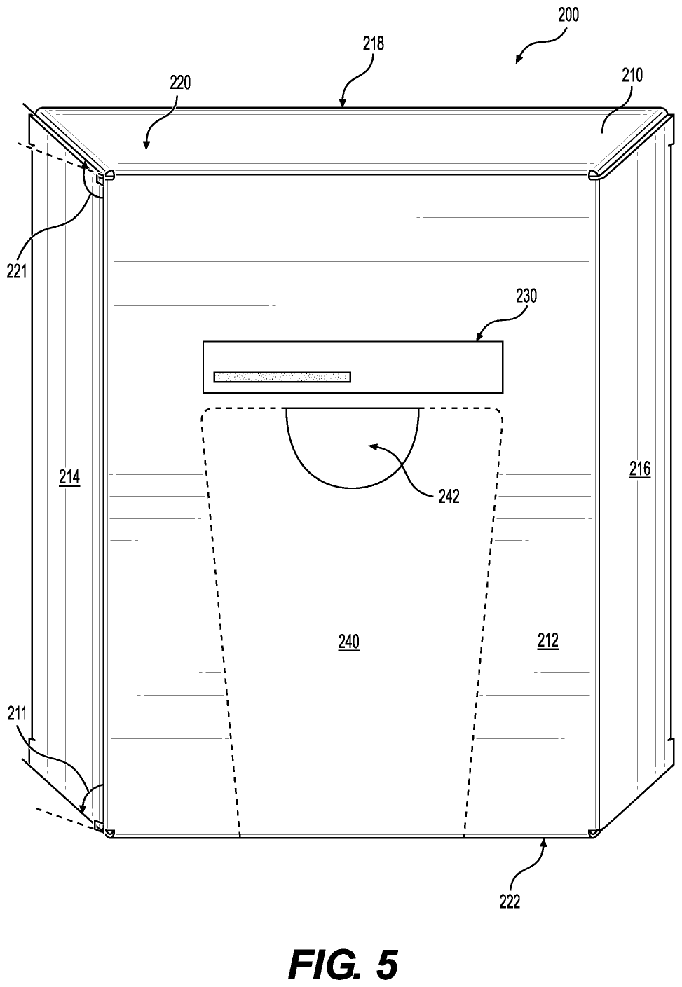

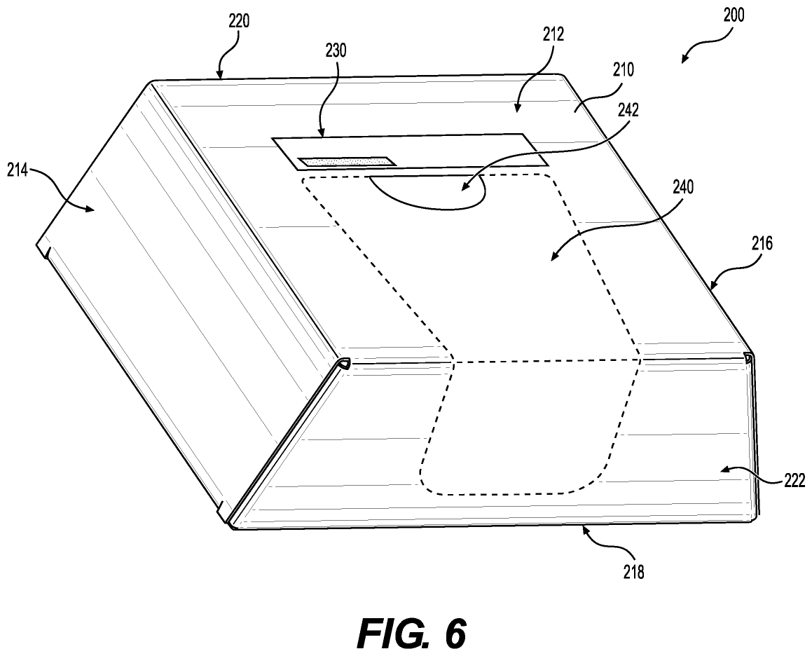

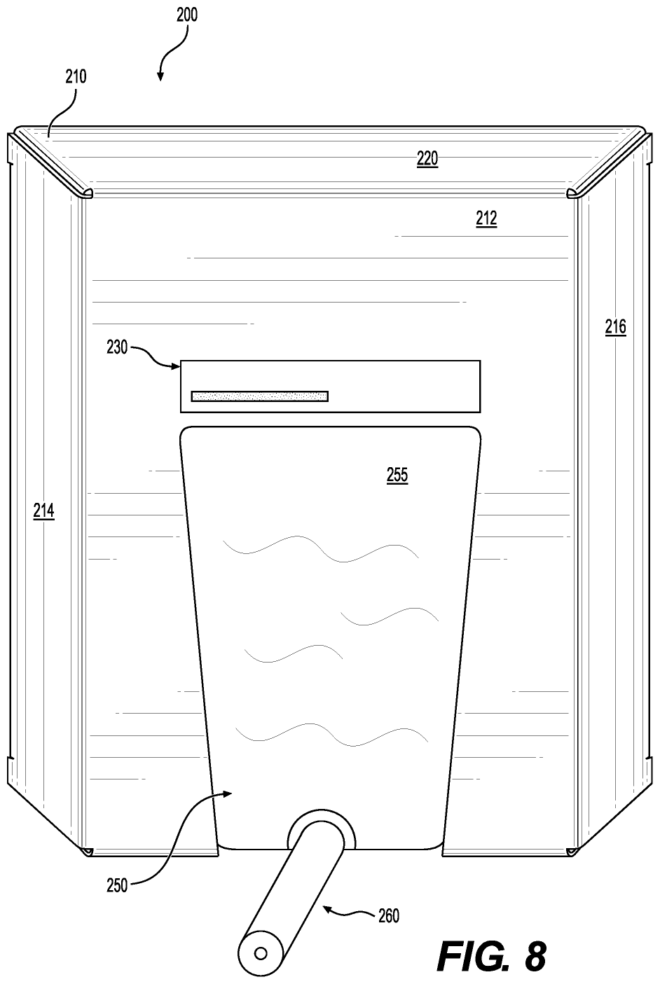

With additional reference to FIGS. 5 to 8, the cartridge 200 will now be described in more detail.

The cartridge 200 includes a cartridge housing 210 formed from a sheet of stiff paper-product (cardstock). It is contemplated that the housing 210 could be made from a variety of materials, including but not limited to: cardboard, rigid or semi-rigid plastic, and metal. The housing 210 is formed from a single continuous piece of cardstock, but in some embodiments the housing 210 could be formed from two or more portions connected together.

The cartridge housing 210 includes a front wall 212, a right wall 214 extending rearward from a right edge of the front wall 212, a left wall 216 extending rearward from a left edge of the front wall 212, and a rear wall 218 extending between a rear edge of the right wall 214 and a rear edge of the left wall 216. The rear wall 218 is generally parallel to the front wall 212, such that when the cartridge 200 is in an upright orientation (as installed in the distributor 100), the front and rear walls 212, 218 are generally vertical. The rear wall 218 is wider than the front wall 212, such that the right and left walls 214, 216 extend rearward and outward from the front wall 212. The relative arrangement of the right and left walls 214, 216 to the front and/or rear wall 212, 218 could change depending on the particular embodiment.

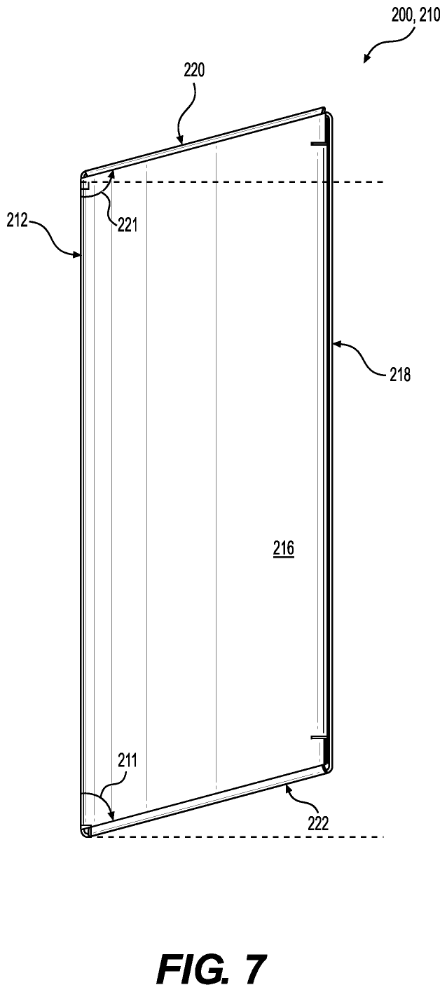

The cartridge housing 210 further includes a bottom wall 222 extending rearward and upward from the front 212 to the rear wall 218. The bottom wall 222 is arranged at an angle 211 of less than 90 degrees to the front wall 212, such that the bottom surface of the cartridge 200 (when the cartridge 200 is oriented upright) is angled forward. See, for example, FIG. 7. In the present embodiment, the angle 211 is approximately 75 degrees. Generally, the angle 211 is contemplated to be greater than 60 degrees; the exact angle 211 chosen for a given embodiment could depend on many factors, including the exact fluid product or the viscosity of the fluid product to the distributed by the distributor 100. The angle 211 is the same as the receiving surface angle 131, such that the bottom wall 222 and front wall 212 of the cartridge housing 210 fit into the recess 132 and are held securely by the receiving surface 130 and the interior of the front wall 112 and the door 120 when the cartridge 200 is installed in the distributor 100. In some cases, the angle 211 and the receiving surface angle 131 could be different or be differently arranged.

The cartridge housing 210 further includes a top wall 220 extending rearward and upward from the front wall 212 to the rear wall 218. The top wall 220 is arranged at an angle 221 of greater than 90 degrees, as is illustrated in FIG. 7. In the present embodiment, the angle 221 is approximately 105 degrees, although different forms are contemplated. The top wall angle 221 is configured to follow the form of the door 120, such that the cartridge 200 fits securely in the recess 132. In some embodiments, the top wall 220 could be differently arranged, such that the top side of the cartridge 200 no longer conforms to the door 120.

The cartridge housing 210 includes a removable portion 240 defined by perforations in the front wall 212 and the bottom wall 222. Depending on the embodiment, the removable portion 240 could be defined in only one of the front wall 212 or bottom wall 222. The removable portion 240 is selectively removable from remaining portions of the cartridge housing 210 in order to access a fluid-containing package 250 disposed therein (described in more detail below). The front wall 212 further defines therein a hole 242 between a top portion of the removable portion 240 and an upper portion of the front wall 212. The hole 242 is configured to allow a user to pull open the cartridge 200 by removing the perforated removable portion 240. In some embodiments, it is contemplated that the removable portion 240 could be omitted and the front wall 212 and/or the bottom wall 222 could define one or more apertures therein.

The cartridge 200 includes the fluid-containing package 250 disposed in the housing 210. The fluid-containing package 250 including a bag 255 containing a fluid product in an interior of the bag 255. The bag 255 is a transparent, plastic enclosure in which the fluid product is sealed. As is mentioned above, the fluid product of the present embodiment is hand-sanitizing gel, although other fluid products are contemplated.

The fluid-containing package 250 includes the dispensing tube 260 (see also FIG. 4) that fluidly communicates with an interior of the bag 255. The dispensing tube 260 is inserted and snapped into to a port formed by the bag 255 (not shown), but other methods of sealably connecting the tube 260 to the bag 255 are contemplated.

Before removal of the removable portion 240, the dispensing tube 260 is contained within the cartridge housing 210. After the removable portion 240 have been torn along the perforations, the fluid-containing package 250 is configured to allow the dispensing tube 260 to be selectively extended outward and downward from the cartridge housing 210. As can be seen from at least FIG. 4, the removable portion 240 is shaped and arranged to allow the dispensing tube 260 to extend generally vertically downward from the bag 255. The removable portion 240 is further configured to allow a level of remaining fluid product in the bag 255 to be visible from an exterior of the cartridge 200.

On the front wall 212 of the housing 210, the cartridge 200 includes identifying information 230 related to the fluid product of the fluid-containing package 250. The identifying information 230 is arranged on the front wall 212 such that the identifying information 230 is visible through the window 125 to an end user when the cartridge 200 is installed in the distributor 100. In the present embodiment, the identifying information 230 includes the type of fluid product contained therein (“Antibacterial Cleaner—For Hands”), the active ingredient (“Ethanol 70%”), and the number of doses contained within the cartridge 200 (“1100 doses”). It is contemplated that the identifying information 230 could include more or fewer types of information. In some cases, for example, the identifying information 230 of the fluid product could include, but is not limited to: a scent or perfume, possible allergens, thickening agent ingredients, vitamins, emollients, and any other information that may be of interest to an end user.

One non-limiting example of installation and replacement of the cartridge 200 in the distributor 100 by an operator will now be generally described. Additional steps may be performed in some cases; it is also contemplated that some steps could be omitted depending on the particular situation.

Beginning with the door 120 of the distributor 100 is the closed position, the operator first unlocks the locking mechanism 128 and moves the door 120 to the open position in order to access the cartridge receiving recess 132. Because the door 120 is selectively secured in the closed position by the locking mechanism 128, only the operator with a key for operating the locking mechanism 128 is able to access the cartridge receiving recess 132. In some embodiment, the door 120 could instead include an anti-tamper mechanism for limiting access to the interior of the distributor 100. In some cases, the door 120 could include a latch mechanism for maintaining the door 120 in the closed position.

If there was previously another cartridge 200 installed in the distributor 100, the operator then pulls the used cartridge 200 upward out of the cartridge receiving recess 132. The used cartridge 200 can then be disposed of; in some cases, directions for recycling or properly disposing of the used cartridge 200 could be provided on the cartridge housing 210.

In order to install the cartridge 200, the removable portion 240 is first removed. To do this, the operator inserts one or more fingers in the hole 242 of the housing 210 and then pulls the removable portion 240 toward the bottom wall 222 of the housing 210. In doing so the perforations defining the removable portion 240 tear, allowing the removable portion 240 to fully dislocate from remaining portions of the housing 210.

With the fluid containing package 250 now exposed through the opening left by removing the removable portion 240, the operator then pulls the dispensing tube 260 outward and downward from the housing 210 until the dispensing tube 260 extends through the space in the bottom wall 222 and is oriented generally parallel to the front wall 212. As can be seen in FIG. 4, the dispensing tube 260 when deployed is disposed generally at a forward portion of the cartridge 200, extending downward from a lowest region of the bottom wall 222.

With the cartridge 200 thus prepared, the operator then inserts the cartridge 200 into the cartridge receiving recess 132. When the cartridge 200 is partially disposed in the distributor 100 (illustrated in FIG. 4), the operator makes sure to pass the dispensing tube 260 through the hole 135 in the receiving surface 130, and into the passage 137.

As is mentioned above, the receiving surface 130 and the bottom wall 222 of the cartridge housing 210 are both arranged at approximately 75 degrees to the vertical front wall 212. The cartridge 200 is thus held in place when installed in the distributor 100 with its front surface generally vertically oriented, the dispensing tube 260 extending downward therefrom. In this arrangement, the bottom surface 222 and the receiving surface 130 tilt a bottom portion of the bag 255 at an angle of approximately 75 degrees, with the dispensing tube 260 arranged at the lowest point of the bag 255, to aid in completely emptying the bag 255. The specific angle at which optimal emptying the bag 255 will occur depends on several factors, including but not limited to the viscosity of the fluid product in the bag 255.

With the cartridge 200 disposed in the cartridge receiving recess 132, the operator finishes installation by closing the door 120 and securing the door 120 in the closed position with the locking mechanism 128. During use by end users of the distributor 100, the operator can monitor the remaining amount of fluid product in the cartridge 200 through the window 125. As the distributor 100 further dispenses a predetermined quantity of fluid product (a “dose”) for each operation of the foot pedal 150 (described more below), the operator can also monitor a number of end users having used the distributor 100 in order to know when the cartridge needs to be replaced. In some embodiments, the distributor 100 could include a counter in order to track the number of end users or doses distributed.

One non-limiting example of using the distributor 100 by an end user will now be generally described, the cartridge 200 having previously been installed in the distributor 100 as described above. The end user can begin by inspecting the identifying information 230 on the cartridge 200, which can be seen through the window 125. In this way, the end user can inspect the information about the fluid product to be distributed. In some cases, the end user may decide not to use the distributor 100 following inspection of the information 230, for example when the fluid product in the cartridge 200 includes a perfume or possible allergen that the end user would prefer to avoid. In cases where an operator has attempted to use a non-complaint cartridge, the end user can also identify that the cartridge in the distributor 100 does not, for instance, present information on the fluid product contained therein. In such a case, the end user can also stop before using the unknown fluid product.

If, after inspecting the information 230, the end user decides to continue, the end user then places one or both of their hands in the hand receiving recess 140, below the passage 137 and the dispensing tube 260 disposed therein. A distal end of the dispensing tube 260 is disposed approximately flush with the end of passage 137 and an exterior side of the housing 105, although it is contemplated that the end of the dispensing tube 260 could be inside the housing 105 or extend into the hand receiving recess 140. The recess 140 is configured such that the end user's hands need not touch the distributor 100 to be arranged below the dispensing tube 260. The end user then operates the distributor 100 by applying pressure on the foot pedal 150 with their foot. By pivoting the foot pedal 150, cams connected between the pedal 150 and the pressure member 145 within the distributor 100 cause the pressure member 145 to move into the passage 137 to pinch the dispensing tube 260 between the pressure member 145 and a wall of the passage 137. This pinching of the dispensing tube 260 then causes a predetermined quantity of fluid product to be ejected from the bottom end of the dispensing tube 260 onto the hand(s) of the end user. The predetermined quantity of the present embodiment is 0.9 mL, but the exact quantity will depend on the specific embodiment and the content of the fluid product. For example, in some cases the temperature of the fluid product could impact the amount distributed.

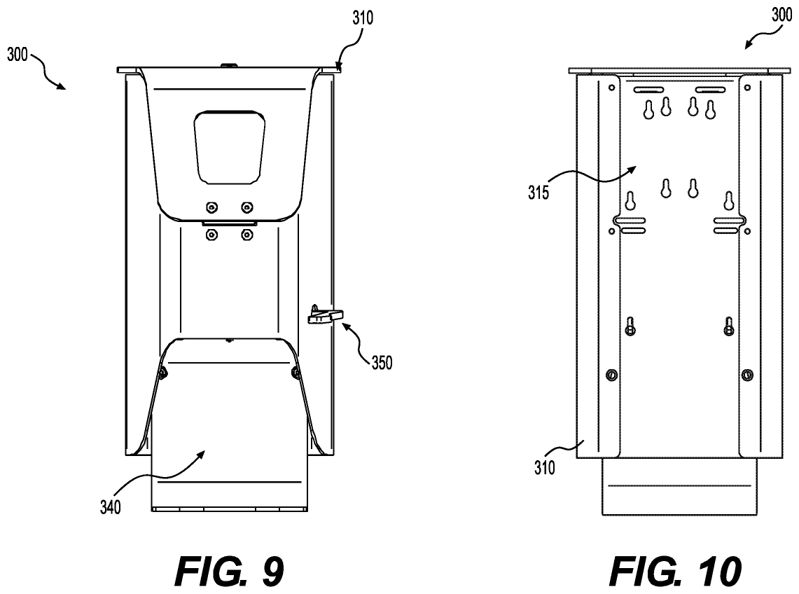

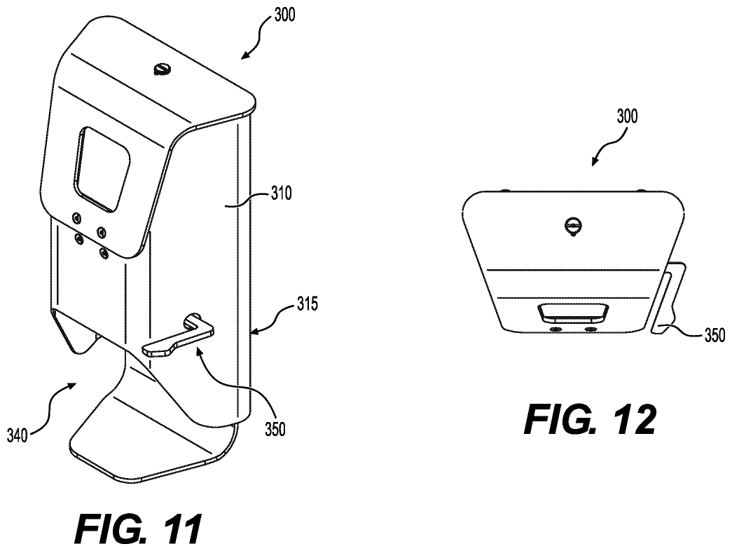

With reference to FIGS. 9 to 12, another embodiment of a distributor 300 according to the present technology is described. Elements of the distributor 300 that are similar to those of the distributor 100 retain the same reference numeral and will generally not be described again.

The distributor 300 is formed by a housing 310 and is configured to be wall-mounted, and as such does not include a base. A back wall 315 of the distributor 300 is configured to mount to a plurality of bolts or other fasteners affixed to a wall. The distributor 300 includes an operating member 350 which extends from the housing 310. The operating member 350 is specifically an arm bar or plate 350 for use by an end user's arm or elbow. The bar 350 extends outward and forward from a left side of the housing 310. In some embodiments, the arm bar 350 could extend from elsewhere on the housing 310, including for example a right side or a front side of the housing 310, or extending across from one side to the other. The arm bar 350 is operatively connected to the pressure member 145 within the housing 310 to dispense the fluid product similarly to the distributor 100.

The housing 310 forms a hand receiving space 340 on a forward side of the housing 310 for receiving the end user's hand underneath the main portion of the housing 310. As with the distributor 100, the hand receiving recess 340 is arranged below the passage 137 through which the dispensing tube 260 extends. The distributor 300 thus functions as follows: The end user inserts one hand in the hand receiving space 340. With their arm, hand, fingers, or elbow, the end user then pushes the arm bar 350 generally downward, triggering the pressure member 137 to pinch the dispensing tube 260 causing the fluid product to be dispensed into the hand receiving space 340.

Modifications and improvements to the above-described embodiments of the present technology may become apparent to those skilled in the art. The foregoing description is intended to be exemplary rather than limiting. The scope of the present technology is therefore intended to be limited solely by the scope of the appended claims.

What is claimed is:

1. A cartridge for a fluid distributer, comprising:
a cartridge housing including:
a front wall,
a right wall extending rearward from a right edge of the front wall,
a left wall extending rearward from a left edge of the front wall,
a rear wall extending between a rear edge of the right wall and a rear edge of the left wall, and
a bottom wall extending rearward and upward from the front wall, the bottom wall being arranged at an angle to the front wall, the angle being less than 90 degrees;
a fluid-containing package disposed in the housing, the fluid-containing package including a bag containing a fluid product in an interior of the bag.

2. The cartridge of claim 1, wherein the fluid-containing package further comprises a dispensing tube fluidly communicating with the interior of the bag.

3. The cartridge of claim 2, wherein:
at least one of the front wall and the bottom wall is perforated to allow for selective removal of a removable portion of the at least one of the front wall and the bottom wall; and
when the removable portion is removed, the fluid-containing package being configured to allow the dispensing tube to be selectively extended outward and downward from the cartridge housing.

4. The cartridge of claim 3, wherein the removable portion is defined by perforations of both the front wall and the bottom wall.

5. The cartridge of claim 4, wherein the removable portion is further configured to allow a level of remaining fluid in the bag to be visible from an exterior of the cartridge.

6. The cartridge of claim 1, wherein:
the front wall displays identifying information related to the fluid product of the fluid-containing package; and
the identifying information is arranged on the front wall such that the identifying information is visible to a user through a window when the cartridge is installed in the fluid distributor

7. The cartridge of claim 1, wherein:
the angle is measured between an inner surface of the front wall and an inner surface of the bottom wall; and
the angle is greater than 60 degrees.

8. The cartridge of claim 1, wherein:
the cartridge housing further includes a top wall extending rearward and upward from the front wall, the top wall being arranged at a top wall angle to the front wall; and
the top wall angle is greater than 90 degrees.

9. The cartridge of claim 1, wherein the fluid product is a hand sanitizing gel.

10. A fluid distributor, comprising:
a distributor housing including:
a front wall,
at least one other wall connected to the front wall, and
a door pivotably connected to one of the front wall and the at least one other wall, the door selectively allowing access to an interior of the distributor housing, the door covering a portion of a front side of the housing,
at least one of the front wall and the door including a transparent window;
a receiving surface disposed in the distributor housing, the receiving surface extending rearward and upward from an interior of the front wall,
the front wall, the door, the at least one other wall, and the receiving surface forming a cartridge receiving recess for selectively receiving a fluid-containing cartridge therein, a front surface of the cartridge being visible through the transparent window when installed therein, the front wall and the receiving surface being configured to match a form of the cartridge;
a passage being defined within the distributor housing for receiving a dispensing tube of the cartridge, the passage being in fluid communication with the recess and an exterior of the housing;
a pressure member disposed adjacent to the passage, the pressure member being selectively moveably into and out of the passage; and
an operating member extending at least partially from the housing, the operating member being operatively connected to the pressure member such that the pressure member moves in the passage upon movement of the operating member by a user,
the pressure member being configured to apply pressure to the dispensing tube upon rotation of the operating member when the cartridge is installed in the distributor.

11. The distributor of claim 10, wherein:
the receiving surface is arranged at a surface angle to the front wall; and
the surface angle is greater than 60 degrees.

12. The distributor of claim 10, further comprising a base; and
wherein:
the distributor housing is connected to and extends upward from the base, and
the operating member is a foot pedal extending from a lower portion of the distributor housing.

13. The distributor of claim 10, wherein:
the distributor housing is configured for mounting to a wall;
the operating member is a pivotable member extending from the distributor housing.

14. The distributor of claim 10, wherein:
the receiving surface defines a hole therein, the cartridge receiving recess being fluidly connected to the passage through the hole; and
when the cartridge is installed in the cartridge receiving recess, the dispensing tube of the cartridge extends through the hole and into the passage.

15. The distributor of claim 10, wherein:
the pressure member is configured to translate generally horizontally into and out of the passage in response to pivoting of the operating member by the user; and
when the cartridge is installed in the cartridge receiving recess, translation of the pressure member causes the dispensing tube of the cartridge to be compressed and eject from the dispensing tube a predetermined amount of fluid product from the cartridge.

16. The distributor of claim 10, wherein the at least one other wall comprises:
a rear wall generally parallel to the front wall;
a left wall extending from a left side of the front wall to a left side of the rear wall; and
a right wall extending from a right side of the front wall to a right side of the rear wall.

17. The distributor of claim 16, wherein:
the door is pivotably connected to the front wall;
the transparent window is disposed in the door; and
when in a closed position, the door extends upward, and then rearward and upward from a top edge of the front wall to a top edge of the rear wall.

18. The distributor of claim 16, wherein:
the front wall, the left wall, and the right wall define a hand-receiving recess on an exterior side of the distributor housing;
the passage is further fluidly connected to the hand-receiving recess; and
when the cartridge is installed in the cartridge receiving recess, the dispensing tube extends through the passage.

19. The distributor of claim 10, further comprising the cartridge disposed in the cartridge receiving recess, the cartridge comprising:
a cartridge housing including:
a front wall,
a right wall extending rearward from a right edge of the front wall,
a left wall extending rearward from a left edge of the front wall,
a rear wall extending between a rear edge of the right wall and a rear edge of the left wall, and
a bottom wall extending rearward and upward from the front wall, the bottom wall being arranged at an angle to the front wall;
a fluid-containing package disposed in the housing, the fluid-containing package including:
a bag containing a fluid product in an interior of the bag, and
the dispensing tube fluidly communicating with the interior of the bag; and
wherein:
the receiving surface and the front wall of the distributer housing are arranged to support the cartridge in a generally vertical orientation, the angle between the bottom wall and front wall of the cartridge housing being approximately equal to the angle between the receiving surface and the front wall of the distributor housing.

20. The distributer of claim 19, wherein the receiving surface and the bottom wall of the cartridge housing are arranged at angles chosen to provide optimal flow for a viscosity value of the fluid product contained in the bag of the fluid-containing package.
